(19) United States
(12) Patent Application Publication
(10) Pub. No.: US 2005/0288292 A1
(43) Pub. Date: Dec. 29, 2005

(54) Pharmaceutical formulation and its use in the treatment of inner ear diseases

(76) Inventors: Werner Bieberschulte, Eschen (LI); Klaus Ehrenberger, Vienna (AT)

Correspondence Address: NIXON & VANDERHYE, PC, 901 NORTH GLEBE ROAD, 11TH FLOOR, ARLINGTON, VA, 22203, US

(21) Appl. No.: 10/522,918

(22) PCT Filed: Jul. 30, 2003
(86) PCT No.: PCT/CH03/00523

§ 371 Date: Aug. 22, 2005

(30) Foreign Application Priority Data
Jul. 31, 2002 (CH) 1357/02

(51) Int. Cl. A61K 31/498

(52) U.S. Cl. 514/249

(57) ABSTRACT

A new pharmaceutical formulation, especially for trans-tympanic or intra-transtympanic administration, containing a quinoxalin-2-one compound of the formula (I) in which R1 and R2, independently of one another, are hydrogen, methyl-, ethyl-, propyl-, butyl- or R1 and R2 together are a cyclo-alkyl compound; R3 is methoxy, ethoxy, hydroxy, hydrogen, C1-C4 alkyl or halogen, and n=1, 2 or 3, or a pharmaceutically compatible salt of the afore-mentioned derivatives, and in addition an effective amount of a permeability accelerator of the afore-mentioned quinoxalin-2-one compound, as well as, if necessary, a pharmaceutically compatible a solvent.

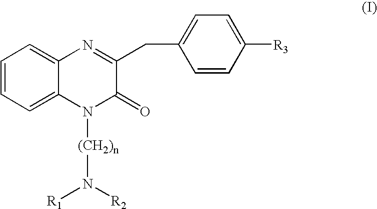

The invention concerns a pharmaceutical formulation as well as the use of this formulation for the treatment especially of inner ear diseases.

Quinoxaline-2-one derivatives have been known since the 60's or early 70's as active pharmaceutical compounds. An important representative of the quinoxaline-2-one derivatives is Caroverin (=non-proprietary name) whose correct chemical name is 1-diethylaminoethyl-3-(p-methoxybenzyl)-1,2-dihydro-quinoxaline-2-one.

EP-A-0 542 689 describes the use of quinoxaline-2-one derivatives for the production of effective pharmaceutical, neuro-protective compounds for the treatment of neuro-toxicities and functional disturbances of the central nervous system. According to EP-A-0 542 689, Caroverin is especially useful in the treatment of cochlear post-synaptic tinnitus. The previously described, pharmaceutically effective quinoxaline-2-one derivatives should be able to be administered orally, rectally or parenterally. In a clinical trial on the treatment of cochlear post-synaptic tinnitus, the patient was administered between 70 and 160 mg of Caroverin dissolved in 100 ml of physiological saline, intravenously.

WO 99/66931 proposes the use of quinoxaline derivatives for the treatment of diseases caused by the presence of free-radicals of cell-oxygen metabolism, for stimulation of nerve cell synthesis and for antagonizing glutamate receptors, etc. According to WO 99/66931, the quinoxaline derivates referred to can be administered by any known method, especially oral, trans-dermal, topical and parenteral, although the intra-venous route is preferred. The topical administration of Caroverin in the form of an ointment has been described for the alleviation of sun injuries as well as of skin inflammation caused by oxidation. For these indications, it is a matter of treating superficially and locally with the active medication, which need not necessarily penetrate the blood circulation system for the activity to be effective. In the example of its use mentioned in WO 99/66931, Caroverin was administered once or more frequently to the patient intravenously in relatively high doses. The dosage used was between 160 mg to 1000 mg per day. For many indications, the degree of effectiveness of Caroverin was strongly dependent on the dosage given, and was only satisfactory at high doses. For example, to achieve a neuro-regenerative effect, it was necessary to administer higher doses of Caroverin.

In view of the very high doses required, the specialists did not previously consider the administration of Caroverin by a trans-dermal route, since with trans-dermal administration, it is well known that only relatively small active amounts may be introduced into the blood stream. Trials undertaken by the inventor have shown also that Caroverin only has a limited permeability through the skin.

In addition, new trials by the inventor have shown that Caroverin is not indicated for the treatment of all forms of tinnitus. Apart from cochlear post-synaptic tinnitus, which, according to EP 0 542 689, is treatable by infusion of Caroverin, it has been observed that a muscular tinnitus (also known as myognathic tinnitus) is not responsive to the intravenous administration of Caroverin. Muscular or myognathic tinnitus is identified by imparting an electrical charge to the chewing musculature, whereby the intensity and frequency of the symptoms of tinnitus can be altered. Myognathic tinnitus can substantially be caused to disappear when the tendon bundles of the middle ear musculature that operates the chain of auditory ossicles are surgically sectioned (tenotomy). The precise mechanism involved in this effect is still unclear.

Morbus Menière, named after the French doctor of the same name, is a relatively rare disease, the symptoms of which are turning-giddiness, ringing in the ears and noise-sensitive hearing deficiency. The disease appears from time to time, and is usually accompanied by severe nausea and vomiting. After some time, a pan-cochlear loss of hearing develops. At the present time, the recommended medical therapies for Morbus Ménière are highly unsatisfactory.

It is, therefore, an object of this invention, to make available an improved pharmaceutical preparation or formulation for the treatment of inner ear diseases. An object of this invention is to provide an improved formulation that can also be administered by the patient himself. Yet another object is to make available a medicine and/or a pharmaceutical formulation for the treatment also of the so-called muscular or myognathic tinnitus, Morbus Ménière, labyrinthine vertigo and impairment of hearing, especially such associated with speech comprehension deficiency.

According to the present invention, the object is achieved by means of a pharmaceutical formulation, especially for trans-tympanic or intra-transtympanic administration, that contains a quinoxalin-2-one derivative of the formula 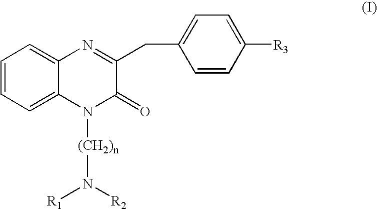
in which R₁ and R₂ independently of one another, are hydrogen, methyl-, ethyl-, propyl- or butyl-, or R1 and R2 together form a cyclo-alkyl compound.

R3 is methoxy, ethoxy, hydroxy, hydrogen, C1-C4 alkyl or halogen;

and n=1, 2 or 3, or a pharmaceutically compatible salt of the aforesaid derivatives; and, further containing an active amount of a compound which, with reference to the above-mentioned quinoxalin-2-one derivatives works as a permeability accelerator or carrier in respect of the afore-mentioned quinoxalin-2-one derivatives; as well as, if required, a pharmaceutically compatible solvent.

The inventor was surprised to discover that, with the help of a suitable permeability accelerator, or activator, and carrier system, respectively, a formulation could be identified that could provide trans-tympanic administration, and that was already highly effective with low dosages. This result was completely unexpected since it had been necessary to administer relatively high doses of quinoxalin-2-one derivatives intravenously in the case of the abovementioned indications. Therefore, it could not have been expected that the quinoxalin-2-one derivatives together with a suitable permeability accelerator or carrier system were already effective at much lower dosages when the novel formulation was applied through the tympanum.

Especially effective are the quinoxalin-2-one derivatives in which the R1 and R2 substituents are an ethyl group, where n=2, and in which R3 is a methoxy group, so that the molecule is 1-diethylaminoethyl-3-(p-methoxybenzyl)-1,2-dihydro-quinoxalin-2-one (non-proprietary name: Caroverin) or a pharmaceutically compatible salt thereof. Furthermore, quinoxalin-2-one derivatives are also effective in which the R1 and R2 substituents are an ethyl group where n=2, and R3 is a hydroxy group, so that the molecule is 1-diethylaminoethyl-3-(p-hydroxybenzyl)-1,2-dihydro-quinoxalin-2-one or a pharmaceutically compatible salt thereof.

Advantageously, the permeability accelerator used in the formulation is one of the following compounds: dimethyl sulphoxide, mono-glyceride, ethyl- or methyl-palmitic acid ester, fatty acids, fatty acid esters, fatty acid alcohols, substituted dialkyl fatty acids having 8 to 14 carbon atoms, N-methyl pyrrolidone, N-methyl-2-pyrrolidone oleic acid, propylene glycol, diethylene glycol, the mono-alkyl ether or carboxy-methyl ether of poly-ethylene glycol, propylene glycol fatty acid ester, lauryl acetate, N,N-dialkyl laurarnide, histamine, a dialkyl lauramide/dimethyl formamide mixture, dimethyl acetamide, N,N-diethyl-m-toluamide, ethylene glycol monomethyl ether, isopropyl myristate, isopropyl palmitate, propylene glycol and oleic acid or oleyl alcohol, 2-pyrrolidone and dimethyl formamide, lauric acid, linoleic acid, lauryl acetate, sodium oleate, glycerine mono-oleate, urea, 1-bisabolol. Also possible is the use of a combination of 2 or more of the aforementioned permeability accelerators.

In a preferred formulation, the permeability accelerator used is at least dimethyl sulphoxide or propylene glycol. It has been shown that dimethyl sulphoxide is a good solvent and, surprisingly, a good permeability accelerator for quinoxalin-2-one derivatives, especially for Caroverin. Furthermore, dimethyl sulphoxide can be added in fairly high concentrations. The content by weight of dimethyl sulphoxide in the formulation preferably comprises between 5 and 50%, but can also be higher. Advantageously, the formulation contains, together with dimethyl sulphoxide, at least another second permeability accelerator. This can be a member of the above-mentioned group of permeability accelerators. By the addition of at least one further permeability accelerator, the content by weight of dimethyl sulphoxide can be correspondingly reduced. In this way, the risk is reduced that the administration of the formulation causes skin irritation.

Preferably, the second permeability accelerator is a glycol compound such, for example, as an ethylene- and/or propylene glycol compound.

Advantageously, the ratio by weight of quinoxalin-2-one derivative to the permeability accelerator is between 1:2 and 1:500, preferably between 1:20 and 1:100. As the solvent, for example, glycerine or another physiologically compatible compound, such as water, can be used.

An advantageous formulation uses a nanoemulsion or liposomes, which contain the said quinoxalon-2-one compound according to Formula (I), as the permeation accelerator or carrier. Expediently, the nanoemulsion or the liposomes contain a membrane-forming molecule and a coemulsifier besides the said quinoxalon-2-one derivatives.

Substances which make it possible to form two-layer systems (so-called “bilayers”) are preferably used as the membrane-forming molecule. A phospholipid is preferably used as the membrane-forming molecule. It may be a hydrogenated or partially hydrogenated phospholipid. A naturally or artificially produced lecithin is preferably used. The latter may, for example, be obtained from soy beans or hen's eggs. Examples of phospholipids are phosphatidylcholine, phosphatidylethanolamine, phosphatidylglycerol, phosphatidylinositol, phosphatidylserine and sphingomyelin. The acyl chain may be either saturated or unsaturated, and may have from 12 to 22, preferably from 14 to 18 C atoms. Other liposome-forming membrane lipids such as glycolipids, ceramides, gangliosides and cerebrosides may be used instead of, or partially instead of, phospholipids.

The lipids may be derived from natural plant, animal or microbiological sources, synthetically produced or partially synthetically produced, inclusive of monoacyl phospholipids derived from polyethylene glycol (PEG), for example pegylated monoacyl phosphatidylethanolamine.

According to a particularly preferred embodiment, a phospholipid of the formula 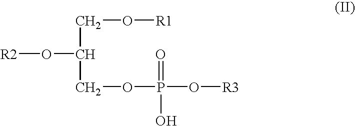
in which

R1 denotes C₁₀-C₂₀ acyl;

R2 denotes hydrogen or C₁₀-C₂₀ acyl

R3 denotes hydrogen, 2-trimethylamino-1-ethyl, 2-amino-1-ethyl, unsubstituted C₁-C₅ alkyl or C₁-C₅ alkyl substituted with one or more carboxyl, hydroxy or amino groups; the inositol or glyceryl group, or salts of these compounds, is used as the membrane-forming molecule.

The C₁₀-C₂₀ acyl is preferably a straight-chained C₁₀-C₂₀ alkanoyl having an even number of C atoms or straight-chained C₁₀-C₂₀ allcanoyl having one or more double bonds and an even number of C atoms. Straight-chained C₁₀-C₂₀ alkanoyls having an even number of C atoms are, for example, n-dodecanoyl, n-tetradecanoyl, n-hexadecanoyl or n-octadecanoyl. Straight-chained C₁₀-C₂₀ alkanoyls having a double bond and an even number of C atoms are, for example, 6-cis- or 6-trans-, 9-cis- or 9-trans-dodecenoyl, -tetradecenoyl, -hexadecenoyl, -octadecenoyl or -icosenoyl, in particular 9-cis-octa-decenoyl (oleoyl), and 9,12-cis-octadecadienoyl or 9,12,15-cis-octadecatrienoyl.

A phospholipid of Formula (II) in which R3 denotes 2-trimethylamino-1-ethyl is referred to by the trivial name lecithin, and a phospholipid of Formula (II) in which R3 denotes 2-amino-1-ethyl is referred to by the trivial name cephalin. For example, naturally occurring cephalin or lecithin having different or identical acyl groups, or mixtures thereof, are suitable.

The membrane-forming component is preferably used in a concentration of about 0.1 to 30% by weight, expressed in terms of the total weight of membrane-forming component, emulsifier and active substance.

One of the following coemulsifiers, or an emulsifier mixture of two or more of the coemulsifiers listed below, may be used as the emulsifier:

Alkali metal, ammonium and aminium salts of fatty acids, for example lithium, sodium, potassium, ammonium, triethylamine, ethanolamine, diethanolamine or triethanolamine salts. Sodium, potassium or ammonium (NR₁R₂R₃) salts are preferred, R₁, R₂ and R₃ independently of one another denoting hydrogen, C₁-C₄ alkyl or C₁-C₄ hydroxyalkyl. Alkyl sulfates such as, for example, sodium dodecyl sulphate.

Salts of bile acid, for example sodium cholate, sodium glycocholate and sodium taurocholate;

partial fatty acid esters of sorbitan, for example sorbitan monolaurate;

sugar esters, for example sucrose monolaurate;

fatty acid partial glycerides, for example lauric acid monoglyceride;

polyglycerol esters of fatty acids;

propylene glycol esters of fatty acids;

lactic acid esters of fatty acids, for example sodium stearoyl-lactyl-2-lactate;

proteins, for example casein.

Emulsifiers of the polyoxyethylene type are more particularly preferred. Examples of such emulsifiers are:
polyethoxylated sorbitan fatty acid esters, for example polysorbate 80;
polyethoxylated vitamin E derivates, for example vitamin E polyethylene glycol 1000 succinate;
polyethoxylated fatty acid partial glycerides, for example diethylene glycol monostearate;
polyethoxylated carbohydrates;
block polymers of ethylene oxide and propylene oxide, for example poloxamer 188.

The emulsifier is advantageously present in the formulation at a concentration of about 1 to about 50% by weight, expressed in terms of the total weight of the membrane-forming component, the emulsifier and the active substance. The quinoxalin-2-one compound is present in the formulation at a concentration of about 0.1 to 70% by weight, expressed in terms of the total weight of the components (membrane-forming component, emulsifier and quinoxalin-2-one compound).

The subject of the present invention also comprises the use of a quinoxalin-2-one derivative of the formula: 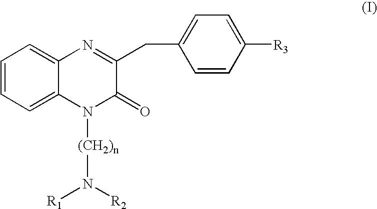
in which R1 and R2, independently of one another, are hydrogen, methyl-, ethyl-, propyl- or butyl-, or R1 and R2 together form a cyclo-alkyl compound;

R3 is the methoxy, ethoxy, hydroxy, hydrogen, a C1-C4 alkyl or halogenatom, and n=1, 2 or 3;

or a pharmaceutically compatible salt of the afore-mentioned quinoxalin-2-one compound; and

an effective amount of a compound that is a permeability accelerator, also in the form of a suitable carrier like a nanoemulsion or liposomes, in respect of the above-mentioned quinoxalin-2-one derivatives for the manufacture of a pharmaceutical formulation that can be administered through the tympanum for the treatment of inner-ear diseases. Preferably, Caroverin or a salt compound of Caroverin is used in the formulation. Salt compounds have the advantage that they are more readily soluble in divers solvents.

A further subject of the present invention is the use, in accordance with the claims 30 to 37, for the treatment of the hitherto unknown indications, such as the treatment of myognathic tinnitus, Morbus Ménière, labyrinthine vertigo or speech discrimination impediments together with hearing impediments. These are new indications for quinoxalin-2-one derivatives and, in particular, for Caroverin. For the aforementioned indications the quinoxalin-2-one derivatives can be used also in accordance with a known form of administration like, e.g. intravenous injection. Preferably, the quinoxalin-2-one derivatives, however, are administered in a formulation together with a permeability accelerator or in a carrier system in the form of liposomes or a nanoemulsion according to the above description.

The formulation according to the invention is preferably administered in a liquid form. Aqueous formulations as well as non aqueous ones can be used. Advantageously the viscosity of the formulation is between 5000 and 25000 mPas (milli-Pascal per second), preferably between 15000 and 20000 mPas, so that a longer period of administration of the active ingredient into the inner ear is achieved. The part by weight of quinoxalin-2-one derivative used is preferably between about 0.5% and 12%.

The treatment of a patient takes place as follows: An absorbable material such, for example, as a wick of about 2 mm diameter and 2 to 3 cm in length is soaked with the inventive formulation. The soaked wick is then inserted into the ear so that it lies against the tympanic membrane. Depending on the concentration of the solution used, the therapy lasts between 3 and 24 hours. Depending on the condition of the disease, the above described treatment is continued by the additional administration of a specific number of drops of the active formulation, such, for example, as 3-4 drops every second day.

In the case where the formulation is a nanoemulsion, the formulation can also be applied directly onto tympanum.

Alternatively, the formulation can also be administered intra-trans-tympanically. For this form of administration of active substances, a canula or drainage tubule that reaches into the middle ear, is used and the active substances are administered via the drainage tubule.

### EXAMPLES OF THE FORMULATION

Three examples of the non-aqueous formulations are as follows:

### Example 1

0.5% of Caroverin

20% of dimethyl sulphoxide

30% of propylene glycol

50% of glycerine

### Example 2

0.5% of Caroverin

20% of dimethyl sulphoxide

30% of propylene glycol

50% of gel

### Example 3

10% of Caroverin hydrochloride

20% of dimethyl sulphoxide

30% of propylene glycol

40% of glycerine

### Example 4

1% of Caroverin

3% of acetone

96% of propylene glycol

The viscosity of the different trial solutions (formulations) is, preferably, between 5000 and 25000 mPas, more preferably between 15000-20000 mPas.

The following example is of an aqueous formulation with a thickening agent:

### Example 5

1% of Caroverin hydrochloride

20% of dimethyl sulphoxide

30% of propylene glycol

48% of water

1% of thickening agent (PVMIMA decadiene cross-polymer).

The viscosity of the solution is preferably increased by the addition of a thickening agent, so that, during the drop-wise administration of the solution into the outer auditory meatus the solution stays as long as possible on the tympanic membrane without running out. Because of the good adhesion of the formulation on the tympanic membrane, a long lasting administration of the active ingredient into the inner ear is ensured.

Preparation of a Nanoemulsion/Liposomes

In order to produce the formulation, the emulsifier and the quinoxalin-2-one compound are mixed to form a homogenous liquid phase, optionally while heating. The phospholipid is dissolved in this phase, optionally with the aid of a solubility promoter, for example ethanol. This results in a homogenous solution. It may then be diluted with water to the intended concentration of active substance.

1. A pharmaceutical formulation, especially for the trans-tympanic or intra-transtympanic administration, according to which the formulation contains a quinoxalin-2-one derivative of the formula 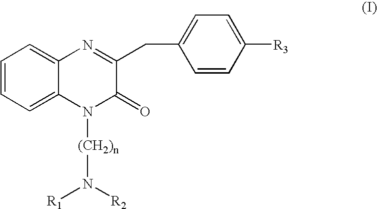
in which R1 and R2, independently of one another, are hydrogen, methyl-, ethyl-, propyl- or butyl- groups, or R1 and R2 together form a cyclo-alkyl compound, R3 is methoxy-, ethoxy-, hydroxy-, hydrogen, C1-C4-alkyl- or halogen; and n=1, 2 or 3;
or a pharmaceutically compatible salt of the aforesaid derivatives; and, in addition, containing an effective amount of a compound that acts as a permeability accelerator or carrier in respect of the afore-mentioned quinoxalin-2-one derivatives; as well as, if necessary, a pharmaceutically compatible solvent.

2. A pharmaceutical formulation as claimed in claim 1, according to which R1 and R2 are ethyl groups; n=2, and R3 is a methoxy group, so that the molecule is 1-diethylaminoethyl-3-(p-methoxybenzyl)-1,2-dihydro-quinoxalin-2-one (INN: Caroverin), or a pharmaceutically compatible salt thereof.

3. A pharmaceutical formulation as claimed in claim 1, according to which R1 and R2 are ethyl groups; n=2; and R3 is a hydroxy group, so that the molecule is 1-diethylaminoethyl-3-(p-hydroxy-benzyl)-1,2-dihydro-quinoxaline-2-one or a pharmaceutically compatible salt thereof.

4. A pharmaceutical formulation as claimed in claim 1, according to which the permeation accelerator or carrier comprises at least one of the following compounds: Dimethyl sulphoxide, monoglyceride, ethyl- or methyl-palmitic acid ester, fatty acids, fatty acid esters, fatty acid alcohols, substituted dialkyl fatty acids having 8 to 14 carbon atoms, N-methyl-pyrrolidone, N-methyl-2-pyrrolidone, oleic acid, propylene glycol, diethylene glycol, the monoalkyl ether or carboxy-methyl ether of polyethylene glycol, propylene glycol fatty acid ester, lauryl acetate, N,N-dialkyl lauramide, N,N-dialkyl lauramide/dimethyl formamide mixture, dimethyl acetamide, N,N-diethyl-m-toluamide, histamine, ethylene glycol monomethyl ether, isopropyl myristate, isopropyl palmitate, propylene glycol and oleic acid or oleic alcohol, 2-pyrrolidone and dimethyl formaniide, lauric acid, linoleic acid, lauryl acetate, sodium oleate, glycerine mono-oleate, urea and 1-bisabolol.

5. A pharmaceutical formulation as claimed in claim 1, according to which the permeability accelerator used at least contains dimethyl sulphoxide or propylene glycol.

6. A pharmaceutical formulation as claimed in claim 1, according to which the part by weight of dimethyl sulphoxide in the formulation is between 5 and 50%.

7. A pharmaceutical formulation as claimed in claim 1, according to which at least one further, second permeability accelerator is contained in combination with dimethyl sulphoxide.

8. A pharmaceutical formulation as claimed in claim 1, according to which the second permeability accelerator is a glycol compound.

9. A pharmaceutical formulation as claimed in claim 7, according to which the second permeability accelerator is ethylene- or propylene glycol.

10. A pharmaceutical formulation as claimed in claim 1, according to which the ratio by weight of the quinoxalin-2-one derivative to the permeability accelerator is between 1:2 and 1:500, preferably between 1:20 and 1:100.

11. A pharmaceutical formulation as claimed in claim 1, according to which glycerine and/or water are used as the solvent.

12. A pharmaceutical formulation as claimed in claim 1, according to which the viscosity of the formulation is between 5000 and 25000 mPas (milliPascal), preferably between 15000 and 20000 mPas.

13. A pharmaceutical formulation as claimed in claim 1, according to which a nanoemulsion or liposomes, which contain the said quinoxalon-2-one compound according to Formula (I), are used as a permeation accelerator or carrier.

14. A pharmaceutical formulation as claimed in claim 13, according to which the nanoemulsion or the liposomes contain the following compounds besides the said quinoxalon-2-one compound:
a membrane-forming molecule and
a coemulsifier.

15. The use of a quinoxalin-2-one compound of the formula 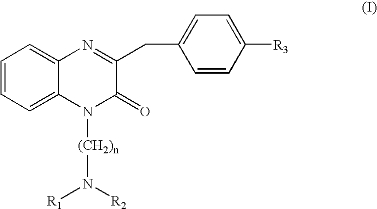
according to which R1 and R2, independently of one another, are hydrogen, methyl-, ethyl-, propyl- or butyl-, or R1 and R2 together form a cyclo-alkyl compound;
R3 is methoxy, ethoxy, hydroxy, hydrogen, C1-C4 alkyl or halogen; and n=1,2 or 3,
or a pharmaceutically compatible salt of the afore-mentioned quinoxalin-2-one compound, together with an effective amount of compound that acts as a permeability accelerator or carrier in respect of the quinoxalin-2-one compound, for the production of a pharmaceutical formulation for trans-tympanic or intra-trans-tympanic administration.

16. The use as claimed in claim 15, according to which R1 and R2 are ethyl groups, n=2 and R3 is a methoxy group, so that the molecule is 1-diethyl-aminoethyl-3-(p-methoxybenzyl )-1,2-dihydro quinoxalin-2-one or a pharmaceutically compatible salt thereof.

17. The use as claimed in claim 15, according to which R1 and R2 are ethyl groups, n=2 and P3 is a hydroxy group, so that the molecule is 1-diethyl-aminoethyl-3-(p-hydroxybenzyl)-1,2-dihydro quinoxalin-2-one or a pharmaceutically compatible salt thereof.

18. The use as claimed in claim 15, according to which the permeability accelerator at least contains dimethyl sulphoxide or propylene glycol.

19. The use as claimed in claim 18, according to which the part by weight of dimethyl suphoxide used in the formulation is between 5 and 50%.

20. The use as claimed in claim 15, according to which at least one further second permeability accelerator is contained in combination with dimethyl sulphoxide.

21. The use as claimed in claim 20, according to which the second permeability accelerator used is a glycol compound.

22. The use as claimed in claim 20, according to which the second permeability accelerator used is ethylene- and/or propylene glycol.

23. The use as claimed in claim 15, according to which the ratio by weight of quinoxalin-2-one to the permeability accelerator is between 1:2 and 1:500, preferably between 1:20 and 1:100.

24. The use as claimed in claim 15, according to which the solvent used is glycerine and/or water.

25. The use as claimed in claim 15, according to which a nanoemulsion or liposomes, which contain the said quinoxalon-2-one compound according to Formula (I), are used as a permeation accelerator or carrier.

26. The use as claimed in claim 25, according to which the nanoemulsion or the liposomes contain the following compounds besides the said quinoxalon-2-one compound:
a membrane-forming molecule and
a coemulsifier.

27. The use as claimed in claim 15, according to which the formulation is liquid and the part by weight of the quinoxalin-2-one compound is between 0.5% and 12%.

28. The use as claimed in claim 15, according to which the formulation is used either as a non-aqueous or as an aqueous formulation.

29. The use as claimed in claim 15, according to which it is used for the treatment of inner ear diseases.

30. The use as claimed in claim 15, according to which it is used for the treatment of muscular or myognathic tinnitus.

31. The use as claimed in claim 15, according to which it is used for the treatment of Morbus Ménière.

32. The use as claimed in claim 15, according to which it is used for the treatment of speech-discrimination deficiency, especially in combination with hearing deficiency.

33. The use as claimed in claim 15, according to which it is used for the treatment of labyrinthine vertigo.

34. The use of a quinoxalin-2-one compound of the formula 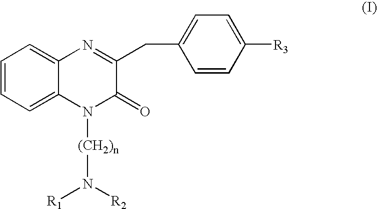
according to which R1 and R2, independently of one another, are hydrogen, methyl-ethyl-, propyl- or butyl-, or R1 and R2 together are a cyclo-alkyl compound; R3 is methoxy, ethoxy, hydroxy, hydrogen, C1-C4 alkyl or halogen; and n=1,2 or 3, preferably Caroverin or a pharmaceutically compatible salt of the afore-mentioned quinoxalin-2-one compound, for the production of a medicine for the treatment of muscular or myognathic tinnitus.

35. The use of a quinoxalin-2-one derivative of the formula 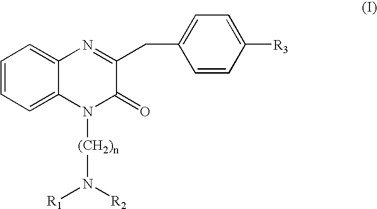
according to which R1 and R2, independently of one another, are hydrogen, methylethyl-, propyl- or butyl- or R1 and R2 together are a cyclo-alkyl compound; R3 is methoxy, ethoxy, hydroxy, hydrogen, C1-C4 alkyl or halogen; and n=1, 2 or 3, preferably Caroverin, or of a pharmaceutically acceptable salt of one of the aforementioned quinoxalin-2-one compounds for the production of a medicine for the treatment of Morbus Ménière.

36. The use of a quinoxalin-2-one derivative of the formula 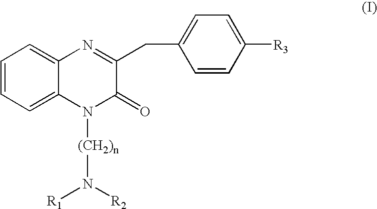
In which R1 and R2, independently of one another, are hydrogen, methyl-, ethyl-, propyl-, butyl-or R1 together with R2 are a cyclo-alkyl compound; R3 is methoxy, ethoxy, hydroxy, hydrogen, C1-C4 alkyl, or halogen; and n=1, 2 or 3, preferably Caroverin, or of a pharmaceutically compatible salt of the aforementioned quinoxalin-2-one derivative for the production of a medicine for the treatment of hearing deficiencies, especially such together with speech comprehension deficiencies.

37. The use of a quinoxalin-2-one derivative of the formula 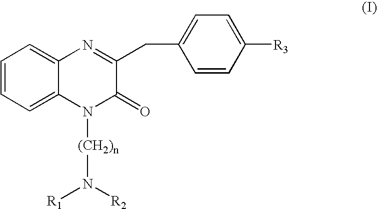
in which R1 and R2, independently of one another, are hydrogen, methyl-ethyl-, propyl-, butyl- or R1 and R2 together are a cyclo-alkyl compound; R3 is methoxy, ethoxy, hydroxy, hydrogen, C1-C4 alkyl, or halogen, and n=1,2 or 3, preferably Caroverin, or of a pharmaceutically compatible salt of one of the afore-mentioned quinoxalin-2-one compounds for the production of a medicine for the treatment of labyrinthine vertigo.

38. The use according to claim 34, characterized in that the derivative is Caroverin.
